(19) United States
(12) Patent Application Publication
(10) Pub. No.: US 2017/0318786 A1
(43) Pub. Date: Nov. 9, 2017

(54) AN ANALYTICAL SAMPLE COLLECTOR

(71) Applicant: Trecento Diagnostics LTD, Guildford, Surrey (GB)

(72) Inventor: Mark Leslie PINDER, Guildford, Surrey (GB)

(21) Appl. No.: 15/531,330

(22) PCT Filed: Nov. 27, 2015
(86) PCT No.: PCT/GB2015/053629

§ 371 Date: May 26, 2017

(30) Foreign Application Priority Data
Nov. 27, 2014 (GB) 1421081.9

(51) Int. Cl. A01K 23/00; A01N 47/28; A01N 59/00; A61B 10/00

(52) U.S. Cl. CPC A01K 23/005; A61B 10/007; A01N 47/28; A01N 59/00; A61B 2503/40

(57) ABSTRACT

An analytical sample collector for collecting animal urine includes a container for containing urine and a collection device for collecting urine. The collection device includes a receiver for receiving urine and a channelling element for channelling urine to the container. An attractant composition emits an odor that attracts animals to urinate on the receiver. Kits and methods obtain an analytical sample of animal urine, and collect and analyze an analytical sample of urine from an animal.

### FIELD OF THE INVENTION

The present invention relates to an analytical sample collector for obtaining a sample of urine from an animal, especially a canine, a kit for assembly into an analytical sample collector for obtaining a sample of urine from an animal, a method of obtaining a sample of urine from an animal and a method of analysing a sample of urine from an animal.

### BACKGROUND

It is useful to collect analytical urine samples from animals, such as canines, cats and other mammals, in order to analyse the samples, e.g. to ascertain whether the urine contains chemical markers associated with medical conditions such as metabolites, pathogens, etc. Various apparatus exists to assist the user in obtaining a sample of urine from animals, such as collection receptacles mounted on handles for positioning under a stream of urine to catch a urine sample. One such apparatus described in WO 2007/126668 comprises an open receiving tray for positioning under a stream of urine and mounted on a handle. A collection chamber is removable mounted at a discharge end of the receiving tray for collecting urine received by the receiving tray. A difficulty with such an apparatus is that it requires the user to accurately position the sampling apparatus in a stream of urine, which can be difficult to achieve as it relies to some extent on the co-operation of the animal. It also relies on the user catching the moment at which the animal urinates, which can be difficult to predict and time consuming for the use. Such apparatus also tend to be messy and/or unpleasant to use as urine tends to miss the sampling apparatus and/or be spilt during collection. Another known type of sampling apparatus are ostomy pouching systems which are strapped to the animal to catch and collect urine from the animal. Whilst such devices may alleviate some of the problems associated with handle mounted receptacle sampling apparatus, they are not straightforward to fit to animals, especially dogs, require the co-operation of the animal and being relatively elaborate items are expensive to produce.

It is known to use scents to encourage animals, e.g. canines, to repeatedly urinate in a particular location. For example EP 2 499 906 discloses a dog urinal for parks, gardens etc., which retains a portion of urine in a container in bottom of the urinal. The smell of urine in the container encourages dogs to urinate on a pipe which forms the body of the dog urinal. ES 2 306 627 similarly discloses a dog urinal for parks, gardens etc., comprising a vertical member for promoting urination and a container below a perforated lid. The container may include attractant compositions such as pheromones or natural odours. EP 0 852 111 discloses yet another fixture for collecting urine from dogs in public places. The fixture includes an attractant means that comprises a vertical post for receiving urine and an attractant composition such as an attractive adhesive that emits an attractive odour. The urine flowing down the vertical post is collected and directed to a container which includes means for processing or treating the urine. Whilst such permanent installations may be useful in encouraging canines to urinate in a particular place, rather than fouling the locality, they are not suitable for collecting samples of urine for analysis which need to be from a single animal and free from contamination.

An object of the present invention is to provide an apparatus for obtaining an analytical sample of urine from animals that alleviates some or all the problems associated with known sampling apparatus. In particular, there is a need for an apparatus which is clean and hygienic to use, does not rely on a user catching a stream of urine as and when it occurs and/or which encourages the animal to co-operate with the sampling process.

### SUMMARY OF THE INVENTION

The present invention uses the natural behaviour of certain animals to urinate in a particular place in response to detecting an odour to encourage an animal to urinate on an analytical sample collector designed to collect an analytical sample of urine, i.e. a sample that is suitable for analysis. Whilst the invention is particularly useful in obtaining analytical samples of urine from canines, especially domestic dogs, it can be used for other medium sized land mammals. For example the invention may be used to collect urine samples from domestic cats or from wild animals such as wild dogs and foxes, members of the Mustelidae family, wild cats and any other mammals that use urine for messaging.

In a first aspect, the present invention provides an analytical sample collector for collecting an analytical sample of animal urine, comprising a container for containing urine; a collection device for collecting urine, the collection device including a receiver for receiving urine and a channelling element for channelling urine to the container; and an attractant composition that emits an odour which attracts animals to urinate on the receiver. In a second aspect the invention provides a kit for obtaining an analytical sample of animal urine comprising: a container for containing urine, a collection device including a receiver for receiving urine and a channelling element for channelling urine to the container, or parts for assembly into such a collection device; and an attractant composition that emits an odour which attracts animals to urinate on the receiver. The attractant composition is optionally supplied in the kit sealed, e.g. sealed in a package. Optionally, the kit includes instructions providing directions as to how to assemble the kit into the analytical sample collector of the first aspect of the invention. In a third aspect, the invention provides a method of collecting an analytical sample of urine from an animal, comprising the step of introducing an animal to the analytical sample collector of the first aspect of the invention and/or the step of assembling the kit of the second aspect of the invention into the analytical sample collector of the first aspect of the invention. In a fourth aspect the invention provides a method of obtaining an analytical sample of animal urine comprising the step of supplying to a user the kit of the second aspect of the invention or the analytical sample collector of the first aspect of the invention, and optionally the further step of receiving from the user a sample of animal urine; or a method of obtaining a sample of animal urine comprising the step of receiving a sample of urine that has been collected in accordance with the method of the third aspect of the invention. In a fifth aspect, the invention provides a method of analysing a sample of urine from an animal, wherein the urine sample has been collected using the method of the third aspect of the invention or wherein the urine sample has been obtained in accordance with the method of the fourth aspect of the invention. In all aspects of the invention, the animal is typically a canine.

It has been found that the analytical sample collector enables a user to obtain an analytical sample of urine from an animal easily with a reduced risk of urine being spilt and without the need to place a sampling apparatus into a stream of urine. It can be used indoors or outdoors, including on rough terrain. Furthermore, the sampling apparatus can be simple and cheap to manufacture so may be disposable.

Advantageously, the container for containing urine is a pot or jar. The container is typically empty prior to use and thus collects the urine in its natural state. Alternatively the container may contain absorbent material which absorbs and retains the urine, such as a gel or fibre material which soaks up the urine. Advantageously, the container and/or collection device does not contain any material, such as a gel, fibre or litter material, which might contaminate or otherwise alter the sample, for example, any material through which the sample is filtered, as such materials can inadvertently remove components from the sample, such as cells, for example bacterial cells. Optionally the container contains a preservative for preserving some or all of the elements of the urine, such as, for example peptides contained in the urine. Preferably, the container is sterile. Preferably the container does not contain any material which alters the chemical composition of the urine and, for example, does not contain reactants or catalysts for transforming constituents of the urine, such deodorising materials, dispersants or emulsifiers, that react with constituents of the urine. Advantageously, in the kit of the second aspect of the invention, the container is supplied clean and empty. The container may, for example, be of a plastics material, such as polypropylene or polyethylene. The container may be made from paperboard, such as coated paperboard having an inert water resistant coating, e.g. a wax coating. The container may be adapted to receive a sealing member, such as a lid, for example a screw cap lid or interference fit lid, to seal collected urine in the container. Alternatively, the container may be adapted to facilitate decanting of collected urine to a storage container. For example the container may include a lip or spout to facilitate pouring of a collected urine sample. The container may be a separate article from the collection device, or the container and the collection device may be combined in a composite article. In alternative embodiments applicable to all aspects of the invention, the analytical sample collector does not comprise a container, which is merely optional. In such embodiments, the collector comprises a collection device for collecting urine, the collection device including a receiver for receiving urine and a channelling element for channelling urine to a container; and an attractant composition that emits an odour which attracts animals to urinate on the receiver.

Advantageously, the attractant composition is present on or in the receiver. Having the attractant composition on or in the receiver part of the collection device has been found to encourage the animal to direct urine directly onto the receiver. Advantageously, the attractant composition is present on and in the receiver in such a way that it does not contaminate the sample. For example, in use, the attractant and the sample preferably do not come into contact. The receiver may for example be impregnated with the attractant composition. Alternatively, the receiver may have an attractant composition on its surface. Advantageously, the receiver is of a laminate or multilayer material and the attractant composition is present between layers of material. Alternatively the attractant composition may be present on or in the receiver in a receptacle that is attached to the main body of the receiver.

It is important that analytical samples are collected in clean and empty equipment to prevent cross contamination. The term “clean” as used herein refers in particular to equipment that does not include traces of urine and/or other chemical substances that dissolved in a urine sample or otherwise alter the composition of a urine sample. In practice it has been found that the use of previously unused equipment to collect analytical samples of urine is a reliable way of avoiding contamination and it more attractive to the user than equipment which must be cleaned, e.g. sterilised, after use and kept clean ready for subsequent use. Advantageously, the analytical sample collector is for single use. Advantageously, the analytical sample collector is disposable. Advantageously, the collection device is for single use. Advantageously, the collection device is disposable. Advantageously the analytical sample collector is unused. Advantageously the analytical sample collector is clinically clean and for example, includes no traces of urine prior to use. A single use, disposable collection device and/or analytical sample collector has been found to be an effective way of preventing contamination of the analytical sample. Advantageously, the collection device and/or analytical sample collector are sterile.

Advantageously, the analytical sample collector is portable. Advantageously, the collection device is a portable article. The analytical sample collector can for example be carried to a suitable location where the animal can be introduced to the analytical sample collector. The analytical sample collector preferable weighs no more than 1 kg, especially no more than 500 g, such as 300 g or less. Advantageously, the collection device is collapsible. A collapsible collection device is easily transported to where it is to be used by a user, thus making it easily portable and/or is easier to ship to a user.

Advantageously, the collection device is constructed of degradable material, especially biodegradable material. The use of degradable material enables the device to be disposed of in an environmentally friendly manner. After use outdoors, degradable collection devices may be left out after use to degrade naturally, there being no imperative to collect up the used collection device after use alleviating the need to touch a used collection device that may retain traces of animal urine. Alternatively, the collection device may be disposed of in compostable waste. Degradable collection devices may also be useful when collecting samples from wild animals negating the necessity to retrieve unused collection devices. Collection devices may, for example, be made from natural fibres.

The collection device of the invention may be of any material or combination of materials. Exemplary materials include paperboard, engineered fibreboard, such as engineered microfibers, plastics materials and polymers. Paperboard has been found to be a particular suitable material for use in constructing the collection device of the invention, especially biodegradable paperboard. Paperboard has been found to be light, disposable and/or inexpensive. The paperboard may be coated or otherwise treated to render it water resistant, for example to prevent the urine sample from soaking into the paperboard in use of the apparatus of the invention. It is also important that compounds do not leach out of the paperboard into the urine sample as such compounds could contaminate the sample and cause problems in the sample analysis. Typically the paperboard included in the collection device of the invention has a weight of at least 100 g/m², especially at least 160 g/m², for example, at least 224 g/m². Stiff paper, also known as card, with a weight of at least 160 g/m² has been found to be particularly suitable. The paperboard may, for example, be made of multiple sheets, e.g. in a corrugated or laminated construction.

Advantageously, the collection device comprises a plurality of panels, e.g. sheets of material, such as sheets of paperboard. A collection device constructed of panels of material has been found to facilitate flat-packing of the collection device when not in use and facilitate delivery to the user. Advantageously, the plurality of panels interlock with each other in the assembled apparatus. Advantageously, the collection device is constructed of interlocking panels. The panels may, for example, comprise slots which enable the panels to slot in into each other on assembly. The panels may include tabs configured to be inserted into corresponding slits in another part of the same panel or in a different panel. Advantageously, at least the receiver portion of the collection device comprises a plurality of panels. Advantageously, the receiver includes at least three, preferably at least four panels.

Advantageously, the collection device is a self-supporting, three-dimensional article. The collection device may, for example comprise panels which slot together in a cross shape, especially a cross shape when viewed from above during use. A cross shape has been found to be a simple way of providing a three-dimensional object. Advantageously, the three-dimensional aspect of the collection device allows it to collect urine from any direction, allowing the device to collect a sample irrespective of the direction that the animal approaches the device or urinates from.

The collection device may, for example, be an elongate member, a portion of which is a channelling member configured to be placed within, or above, container and a further portion of which is a receiver configured to protrude from, or extend above, the container in use. The collection device is optionally a compressible resilient member, such as a coiled spring, a sponge or a foam. The compressible resilient member may be supplied in a kit of a second aspect of the invention compressed within the container. The compressible resilient member may be held in place with a restraining member. On assembling the apparatus of the invention, for example in accordance with a method of the third aspect of the invention, the compressible resilient member is allowed to return to an uncompressed state. In its uncompressed state, the compressible resilient member is advantageously an elongate member.

In use, an upper portion of the collection device typically forms the receiver. In use the channelling element is typically in a lower portion of the collection device than the receiving element. The channelling element may, for example, be located directly beneath the receiver during use of the apparatus of the invention. Urine typically flows downwards under gravity from the receiver to the channelling element in use of the apparatus of the invention. Advantageously, the receiver is mounted over and extends above (i.e. protrudes beyond the top of) the container. Advantageously, the top of the receiver is at least 3 cm, especially at least 4 cm above the top of the container. Preferably, the receiver provides a substantially upstanding surface that projects at least 3 cm, especially at least 4 cm above the top of the container. The upstanding surface may, for example, extend from about 5 cm to about 20 cm above the top of the container, more preferably between about 6 to about 15 cm, or from about 7 to about 12 cm. The presence of an upstanding surface against which to urinate makes the device particularly attractive to animals, such as canines, and provides a surface down which urine can flow.

Advantageously, the collection device further comprises a mounting element for mounting the collection device relative to the container and/or for mounting the collection device on a floor. The mounting element may, for example, comprise a plurality of supports, such as legs. Advantageously, the mounting element comprises three or more legs, such as four legs. The mounting element may support the remainder of the collection device in an elevated position off the ground in use. The mounting element advantageously elevates the collection device to enable the channelling element to be positioned above the container in use. Advantageously, the mounting element comprises supports, for example legs, which, in use, straddle the container. Advantageously the mounting element is arranged such that the channelling element and the receiver are supported over the container. It has been found that the provision of legs that straddle the container protects the container from being knocked over by the animal.

Advantageously, the channelling element of the collection device funnels urine that flows down from the receiver to the container. The channelling element may of any shape that directs the flow of urine from the receiver to the container. For example, the channelling element may be a simple rod along which urine flows. Advantageously the channelling element funnels a flow of liquid. For example, the channelling element advantageously has a wide opening at one end and a narrower opening at another end such that a flow of liquid (e.g. urine) is funnelled by the channelling element. The channelling element may be formed from one or more flaps. The flaps may, for example, fold out from a sheet of material that forms the panels discussed above, e.g. fold out from one or more of a plurality of panels that form the receiver. The flaps may, optionally, be held in place by adhesive and or interference fit. The flaps may include tabs which are interested into slits in the panels. Alternatively the flaps may be folded such that they function to channel urine without the need for additional means to hold them in place other than the fold. Alternatively the channelling member may be a cone shaped member. The channelling member may, for example be formed from a sheet of material curled round into a cone shape. Alternatively the channelling member made be a cone shaped article, for example a material moulded into a cone shape.

Advantageously, the collection device is from about 5 cm to about 20 cm in height, especially from about 6 to about 15 cm in height, such as from about 6 to about 12 cm in height or from about 7 to about 10 cm in height. A collection device of more than about 20 cm in height has been found to be knocked over easily and/or is too tall for use by small canine breeds, especially bitches of smaller canine breeds. An optimum height for a large variety of breeds has been found to be about 5 to 12 cm in height. A range of collection device sizes may be provided, for example, to accommodate different breed sizes. Optionally, a single collection device may have a plurality of sizes marked on it (or perforated into it) so that the user can create a collection device of the most appropriate size for the animal in question. Advantageously, the collection device is at least about 7 cm in height, and includes a receiver having a substantially upstanding surface of at least about 3 cm. Alternatively, the collection device is at least about 11 cm in height, and includes a receiver having a substantially upstanding surface of at least about 5 cm.

The width of the collection device, measured at the widest dimension, is preferably from about 12 cm to about 25 cm, especially from about 15 cm to about 20 cm. A width of at least 12 cm, especially 15 cm has been found to add stability to the collection device to lessen the chances of it being knocked over by the animal. Advantageously, the height of the collection device is no more than twice the width of the collection device, measured at the widest dimension, especially no more than 1.5 times the width of the collection device, such as no more than 1.2 times the width of the collection device, for example no more than the width of the collection device. Preferably the collection device is wider than it is high. Having a high width to height ratio has been found to add stability to the collection device.

As will be understood from the above description, the collection device is typically a unitary article. The collection device is preferably a unitary article comprising at least the receiver and channelling element and also further optional elements such as a monitoring element. The collection device may, for example, be a unitary article formed from multiple components that are attached to one another, e.g. a plurality of interlocked panels.

Compositions for attracting animals, especially canines, to urinate in a particular place are known, e.g. to encourage a canine to urinate in a particular location. For example, it is known to provide attractant compositions on pads to encourage canines to urinate on the pad e.g. to facilitate housetraining of puppies or to encourage canines not to foul other areas (see, for example, US 2012/0152175 and US 2011/0139082). Advantageously the attractant composition present in the apparatus and kits of the present invention comprises one or more of a tar, alkylene, sulphur, perfume and a natural oil, preferably each of a tar, alkylene and a perfume. Advantageously the attractant composition is a natural oil. The tar is typically a heavy petroleum fraction, e.g. the fraction of petroleum that remains when the volatile components have been distilled off. The tar typically comprises petroleum fractions having a boiling point of greater than 500° C. at atmospheric pressure. The tar may, for example, be a pitch, such as asphalt, also known as bitumen. The tar may be a coal tar or other distillation product or a by-product of processing a hydrocarbon. Advantageously, the perfume is a volatile component, e.g. having a vapour pressure of at least 2 kpa, preferably at least 4 kpa, an/or having a boiling pint at atmospheric pressure of 100° C. or less, preferably 85° C. or less. The perfume may, for example include chlorine or a chlorinated compounds that have a chlorine aroma, especially volatile chlorinated compounds. The perfume may, for example, include ammonia, urea or compounds comprising amine or urea groups that have the aroma of ammonia, especially volatile compounds comprising amine or urea groups. The alkylene is typically a C₂to C₆ alkylene, preferably a C₂to C₄ alkylene, such as ethylene, propene, butylene or butadiene, especially ethylene and/or butadiene, or mixtures thereof. The attractant may, for example, comprise heavy petroleum fractions having a boiling point of greater than 500° C.; C₂ to C₆ alkylene; chlorine or a chlorinated compounds that have a chlorine aroma and/or ammonia, urea or compounds comprising amine or urea groups that have the aroma of ammonia; and, optionally sulphur. The attractant may, for example, comprise asphalt; ethylene and/or butadiene; chlorine or a chlorinated compounds that have a chlorine aroma; and, optionally sulphur. The attractant composition may be formulated together with binding agents as part of an adhesive composition suitable for adhering two sheets of material, such as a paperboard material, together.

The attractant composition may include a pheromone, such as a natural pheromone, e.g. produced by the animal species from which a sample of urine is to be collected or a natural pheromone from another species, or an artificial pheromone. For example the attractant composition may include a natural or artificial canine pheromone. Advantageously, the attractant composition does not include a pheromone. Advantageously, the attractant composition does not include a natural pheromone, especially not a natural pheromone of the species from which a sample of urine is to be collected. The use of pheromones, especially natural pheromones of the species from which a sample of urine is to be collected have been found to be problematic. For example, the natural pheromone may only attract animals of one gender, the natural pheromone may trigger an unwanted emotional response in the animal, such as aggression, alarm or insecurity. The response of an animal to a natural pheromone can vary, for example due to the age, gender, social standing, physical or emotional well-being of the animal. As such, natural pheromones have not been found to be particularly effective in encouraging a high proportion of a population of a particular animal species to urinate on the apparatus of the invention.

The attractant composition may include urine, such as natural urine, e.g. produced by the animal species from which a sample of urine is to be collected or a natural urine from another species, or an artificial urine derivative. For example the attractant composition may include an artificial derivative of canine urine. Advantageously, the attractant composition does not include urine. Advantageously, the attractant composition does not include natural urine, especially not a natural urine of the species from which a sample of urine is to be collected. Natural urines typically include pheromones and thus suffer from the problems described above. Furthermore, they may be unhygienic and are unappealing for the user to handle. Artificial urine derivatives, such as urea compounds, have been found to be preferable as they can be engineered to tune their properties. For example, they can be engineered to have a wide appeal to a population of animals of a particular species and/or engineered to be more pleasant to handle, for example having a less unattractive odour to humans than natural urine.

In a second aspect, the invention provides a kit for assembly into the apparatus of the first aspect of the invention. The kit typically comprises a container for containing urine as described above, the collection device as described above or parts for assembly into the collection device as described above, and an attractant composition as described above. The container and the collection device may be separate articles or part of a composite article. The kit may for example include a sheet of material, typically two or more sheets of material, such as sheets of paper or paperboard material, for assembly into the collection device as described above. The sheets of material may, for example, include slots to facilitate the sheets to be interlocked into one another. The sheets may, for example, include flaps which, on folding, create the channelling element of the collection device. In addition to the apparatus of the invention the kit may, for example, include a sealing member for sealing the container, e.g. of the type described above. Optionally, instructions providing directions as to how to assemble the kit into the apparatus of the first aspect of the invention are included in the kit.

Advantageously, in the kit of the second aspect of the invention, the attractant composition is sealed. Sealing the attractant composition has been found to preserve the effectiveness of the attractant composition which may otherwise evaporate or deteriorate such that the level of odour emitted by the assembled apparatus is reduced. The sealing may, for example, be in a package, such as a plastics or foil film sachet or a capsule or ampule, in a compartment, e.g. a compartment with a removable seal, especially a compartment on the receiver, or in an encapsulating material, e.g. in the core of a particle or in a colloidal suspension. On assembling the apparatus of the first aspect of the invention from the kit of the second aspect of the invention, the attractant composition is advantageously unsealed, for example by opening a seal on a package or compartment or releasing the composition from the encapsulating material, e.g. by bursting encapsulating particles. If the attractant composition is supplied in the kit on or in the receiver the entire receiver may be sealed, for example it may be supplied in a plastic bag. If the attractant composition is supplied separately from the receiver it is preferably supplied in a sealed package, for example in a sachet, for application to the receiver. If the attractant composition is supplied on in or a part of the receiver only that part of the receiver may be sealed.

Optionally, the kit may comprise a storage device for storing the sample of urine. The storage device is typically a further container, especially a sealable container. A sample of urine contained in the optional storage device may be shipped to a remote location for analysis. The optional storage device is typically empty prior to use and thus collects the urine in its natural state. Alternatively the optional storage device may contain absorbent material which absorbs and retains the urine, such as a gel or fibre material which soaks up the urine. Optionally the optional storage device contains a preservative for preserving some or all of the elements of the urine, such as, for example peptides contained in the urine. Preferably, the optional storage device is sterile. Preferably the optional storage device does not contain any material which alters the chemical composition of the urine and, for example, does not contain reactants or catalysts for transforming constituents of the urine, such as deodorising materials that react with constituents of the urine. The optional storage device may, for example, be of a plastics material, such as polypropylene or polyethylene.

In a third aspect, the invention provides a method of collecting urine from an animal, comprising the step of assembling the kit of the second aspect of the invention into the apparatus of the first aspect of the invention and/or the step of introducing an animal to the apparatus of the first aspect of the invention. The step of introducing an animal may comprise positioning the apparatus in a place where the animal is likely to encounter the apparatus or actively putting the animal and the apparatus in close proximity with each other, e.g. in the same confined space. The method of the third aspect optionally comprises the step of assembling the apparatus, if not supplied ready assembled, e.g. by a user. The assembly step may comprise unsealing the attractant composition, e.g. opening a package, removing a seal to a compartment, or freeing the composition from an encapsulating material. The assembly step may comprise the step of applying the attractant component to the receiver. Preferably the attractant composition is supplied in the kit on or in the receiver and the receiver or a part of the receiver is within the encapsulation. In such embodiments, the releasing of the attractant composition from the encapsulation may, for example, involve removing the receiver or the part of the receiver containing the attractant composition from its packaging or removing a seal from the compartment of the receiver containing the attractant composition. In embodiments wherein the collection device is formed from panels, the panels may be encapsulated, e.g. in a packaging, and the step of releasing the attractant composition from the encapsulation may involve removing a panel from its encapsulation, e.g. removing it from its packaging. The assembly step may comprise constructing the collection device from component parts supplied in the kit, e.g. interlocking sheets of material and/or folding flaps to form the channelling element. The assembly step may, for example, comprise positioning the collection device relative to the channelling element, e.g. underneath the collection device and below the channelling element. The method typically further comprises the step of the user introducing an animal to the analytical sample collector of the first aspect of the invention. The method typically further comprises the step waiting for the animal to urinate on the receiver. The method advantageously comprises the step of monitoring the analytical sample collector to ascertain when an animal has urinated on the analytical sample collector. Advantageously the analytical sample collector is used to collect urine from a single animal. The method advantageously comprises the step of preventing further more than one animal from urinating on the analytical sample collector. For example, the method of the invention may comprise the step of introducing a single animal to the analytical sample collector. The method may, for example, include the step of removing a sample of urine from the container once a single animal had urinated on the analytical sample collector. In the method of collecting an analytical sample of animal urine of the third aspect of the invention, the analytical sample collector used is advantageously clean and/or previously unused, i.e. not previously used to collect a sample of animal urine. Whilst the method of the third aspect of the invention is preferably used to obtain a sample from a single animal, e.g. in order to enable the urine sample of a single animal to be analysed, the method may also be used to collect urine samples from multiple animals, for example, to enable the mixed urine samples of a particular population of wild animals to be analysed. The method typically further comprises the step of sealing the container containing the urine sample with a sealing member or decanting the urine sample into the optional storage device. Advantageously the step of sealing the container containing the urine sample with a sealing member or decanting the urine sample into the optional storage device is performed after a single animal has urinated on the analytical sample collector. The method of the third aspect of the invention may, for example be a method of collecting an analytical sample of urine from a single animal, comprising the step of introducing an animal to the analytical sample collector of the first aspect of the invention and removing a sample of urine from the container once said single animal has urinated on the analytical sample collector.

In a fourth aspect the invention provides a method of obtaining a sample of animal urine comprising the step of supplying the kit of the second aspect of the invention or the apparatus of the first aspect of the invention, e.g. to a user, and optionally the further step of receiving from the user a sample of animal urine; or a method of obtaining a sample of animal urine comprising the step of receiving a sample of urine that has been collected in accordance with the method of the third aspect of the invention. The method of obtaining a sample of animal urine of the fourth aspect of the invention may comprise the step of collecting a sample of urine in accordance with the method of the third aspect of the invention. Thus, for example, the method of the fourth aspect of the invention may comprise the step of supplying the kit of the second aspect of the invention or the apparatus of the first aspect of the invention and the step of collecting a sample of urine in accordance with the method of the third aspect of the invention.

The user may, for example, be the animal owner or other animal handler, a veterinary practitioner such as a veterinary nurse, or any other person interested in obtaining a urine sample from the animal.

In a fifth aspect, the invention provides a method of analysing a sample of urine from an animal, wherein the urine sample has been collected using the method of the third aspect of the invention or wherein the urine sample has been obtained in accordance with the method of the fourth aspect of the invention. The method of the fifth aspect of the invention optionally comprises the step of collecting urine from an animal in accordance with the method of the third aspect of the invention. The method of the fifth aspect of the invention optionally comprises the step of obtaining a sample of animal urine in accordance with the method of the fourth aspect of the invention. The method of the fifth aspect of the invention optionally comprises the step of providing results of the analysis to an interested party.

The interested party may be a dog owner or other dog handler or a veterinary practitioner. The method of the fifth aspect typically further comprises the step of shipping the sealed container or optional storage device containing the urine sample to an analysis facility. Optionally the method comprises the step of analysing the urine sample. The analysis may analyse DNA contained in the urine sample. The analysis may ascertain the levels markers associated with particular diseases or conditions. The analysis may ascertain the level of particular metabolites in the urine sample, such as glucose levels, for example in order to assist in the diagnosis of a disease or condition, such as diabetes.

### DESCRIPTION OF THE DRAWINGS

FIG. 1 shows a schematic view of a first embodiment of the first aspect of the invention.

FIGS. 2 and 3 show elements of a first embodiment of a kit of the second aspect of the invention.

FIG. 4 shows a schematic view of a second embodiment of the first aspect of the invention.

### DETAILED DESCRIPTION OF THE INVENTION

The invention will now be illustrated by way of example.

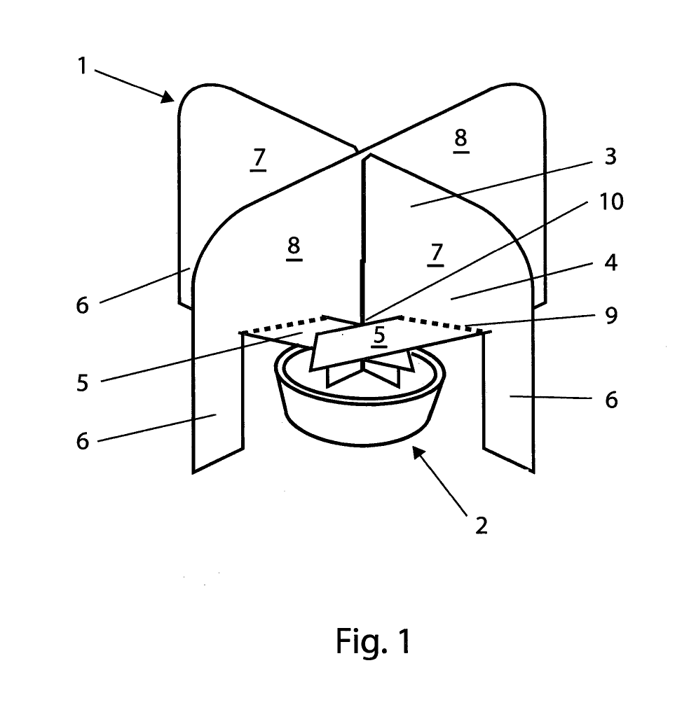

In the embodiment of FIG. 1 the apparatus of the invention comprise a collection device 1 and a container 2 for collecting urine. The collection device 1 comprises in an upper portion a receiver 3 for receiving urine, in a middle portion a channelling element 4 for channelling urine which includes flaps 5 that together form a funnelling arrangement. In a lower portion of the collection device 1 are four legs 6 which together form a mounting element. The collection device 1 is formed of two panels 7, 8, which slot together. The paper panels 7, 8 are coated with a wax such that they resist moisture and allow urine to flow off the collection device 1 rapidly without soaking into the paper. In the embodiment of FIG. 1, each panel 7, 8 is formed from two sheets of stiff paper (not shown) each with a weight of about 200 g/m² that are adhered to one another in all areas except in the area of the flaps 5, such that a separate flap 5 is present on each face of the panels 7, 8. The attractant composition (not shown) is present between the two sheets of stiff paper (not shown) that forms the panels 7, 8. Whilst the flaps 5 may be adhered or otherwise fixed together, in the embodiment shown in FIG. 1, the flaps 5 are folded along the fold lines 9 in such a way that the flaps 5 remain sufficiently close to one another to function to channel urine into the central opening 10.

When the apparatus is in use, an animal attracted by the odour of the attractant composition voluntarily urinates against the receiver 3 of the collection device 1. Urine flows down the wax coated surfaces of the panels 7, 8 and is funnelled by the collection device part to central opening 10. Urine then drips through central space 10 into the container 2.

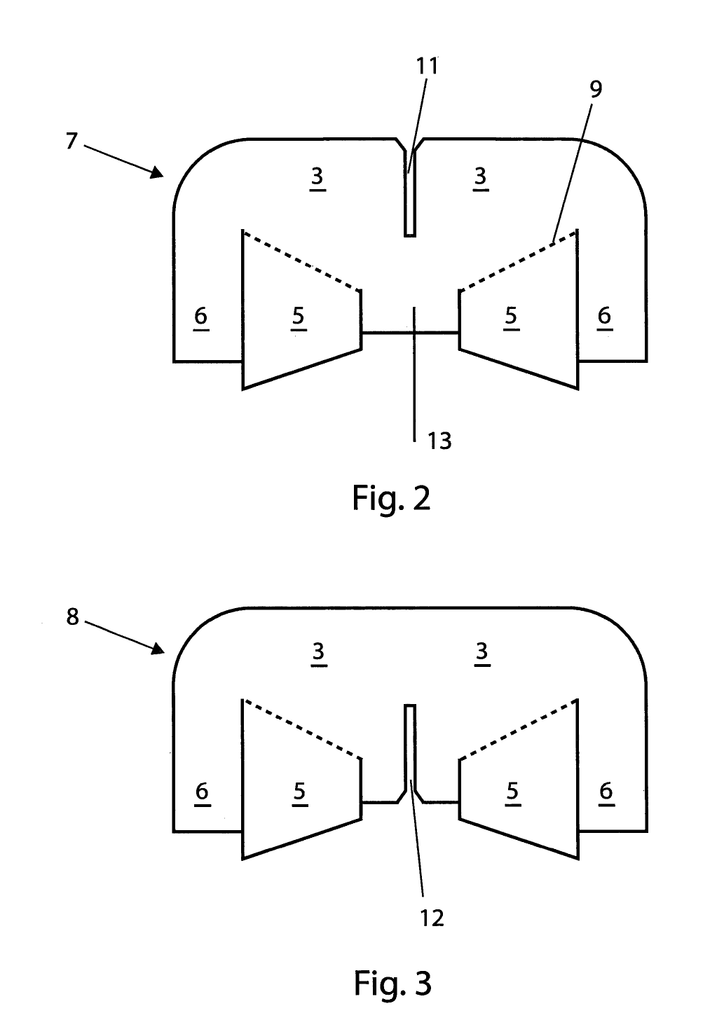

FIGS. 2 and 3 show panels 7, 8, which are elements of an embodiment of a kit of the second aspect of the invention. The panels 7, 8 are each made of two sheets of stiff paper (not shown) each with a weight of about 200 g/m² that are adhered to one another in all areas except in the area of the flaps 5. Panel 7 differs from panel 8 in that panel 7 is provided with a slot 11 in an upper portion of the panel 7, whilst panel 8 is provided with a slot 12 in a lower portion of the panel 8. As such the two panels 7, 8 may be slotted together to form the three dimensional collection device structure 1 shown in FIG. 1. Between the flaps 5 in panel 7 is a tongue portion 13 which in use projects downward below the central opening 10 to direct a flow of urine into the container 2.

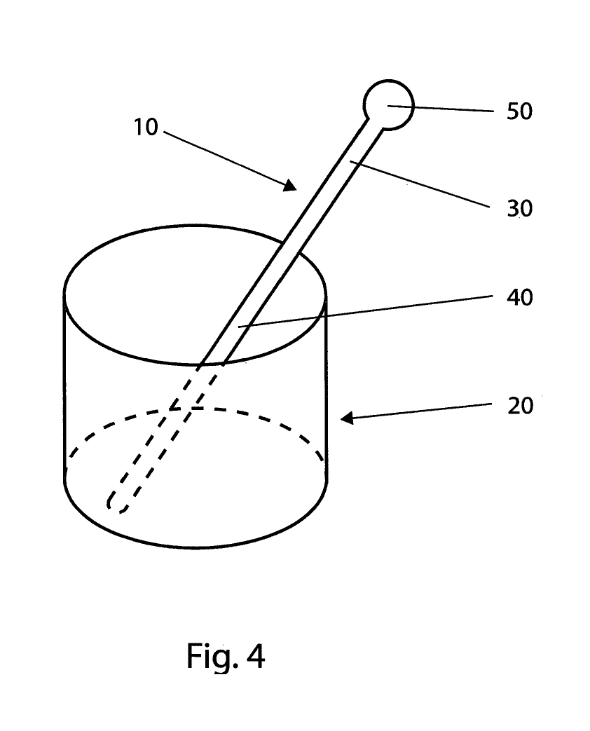

In a second embodiment shown in FIG. 4, the collection device 10 is an elongate member, such as a rod configured to be placed in a container 20 such that an upper portion 30 of the rod protrudes from the container 20, and a lower portion 40 of the rod is within the container 20. The upper portion 30 of the rod is a receiver and the lower portion 40 of the rod is a channelling element. The upper portion 30 of the rod includes a receptacle 50 which contains the attractant composition (not shown). In use, the animal is attracted to urinate on the collection device 10 by to the attractant composition. Urine that is received by the upper portion 30 of the rod flows down the rod and is channelled into the container 20 by the lower portion 40 of the rod.

In a variation of the second embodiment the elongate member that forms the collection device is a resilient member, such as a sponge or a coil. The resilient member is supplied compressed into the container and held in place by a restraining member, e.g. a lid for the jar. On assembling the apparatus, the restraining member is removed such that the resilient member is restored to its uncompressed and elongated state such that it protrudes from the jar. An upper portion of the resilient member, which protrudes from the jar in its uncompressed elongated state, constitutes the receiver and a lower portion of the resilient member, which remains in the jar in its uncompressed elongated state, constitutes the channelling element. The attractant composition is applied to the receiver part of the resilient member. In use urine received by the receiver flows down the resilient member through the channelling element and into the jar.

In an alternative embodiment, the collection device is a truncated cone made of a lightweight plastics material and the collection device is a jar. The attractant composition is in the form of a viscous gel which is supplied in a sachet. In use the truncated cone is placed inverted in the neck of the jar, with the narrow opening inserted into the neck of the jar and the wider opening facing upwards, so as to funnel urine into the collection device. A lower portion of the cone, which includes the narrower opening, constitutes the channelling element of the collection device and an upper portion of the cone, which includes the wider opening, constitutes the receiver. The attractant composition gel is applied to the receiver at an inner surface of an upper portion cone, for example, in a blob. The gel is hydrophobic and has a sufficient viscosity and adherence to the plastics material that it is not washed down into the jar when the animal urinates on the receiver. Once urine has been collected the collection device jar is sealed, e.g. with a lid.

While the invention has been described with respect to exemplified embodiments, these should not be considered as limiting the invention as defined by the following claims.

1. An analytical sample collector for collecting animal urine, comprising:
a container for containing urine;
a collection device for collecting urine, the collection device including a receiver for receiving urine and a channelling element for channelling urine to the container; and
an attractant composition that emits an odour which attracts animals to urinate on the receiver.

2. The analytical sample collector of claim 1, wherein the attractant composition is present on or in the receiver.

3. The analytical sample collector of claim 1, wherein the collection device is a self- supporting, three-dimensional article.

4. The analytical sample collector of claim 1, wherein the collector is disposable and for single use.

5. The analytical sample collector of claim 1, wherein the collector is portable and weighs 1 kg or less.

6. The analytical sample collector of claim 1, wherein the collection device is constructed of paperboard.

7. The analytical sample collector of claim 1, wherein the collection device is constructed of interlocking panels.

8. The analytical sample collector of claim 1 wherein the collection device is an elongate member, a portion of which is a channelling member configured to be placed within the container and a further portion of which is a receiver configured to protrude from the container.

9. The analytical sample collector of claim 8 wherein the elongate member is a compressible resilient member.

10. The analytical sample collector of claim 1, wherein the collection device further comprises a mounting element for mounting the collection device relative to the container such that the channelling element is positioned above the container in use.

11. The analytical sample collector of claim 10, wherein the mounting element comprises three or more legs which, in use, straddle the container such that the channelling element and the receiver are supported over the container.

12. The analytical sample collector of claim 1, wherein the attractant comprises ammonia, urea or an ammonium-based caompound or a urea-based compound.

13. A kit for obtaining an analytical sample of animal urine comprising:
a container for containing urine,
the collection device as defined in claim 1 or parts for assembly into the collection device, and
an attractant composition.

14. The kit of claim 13, further comprising a storage device for storing the sample of animal urine.

15. The kit of claim 13 wherein the attractant composition is sealed.

16. A method of collecting an analytical sample of urine from an animal, comprising the step of introducing an animal to the analytical sample collector of claim 1.

17. The method of claim 16, wherein a sample of urine is collected from a single animal and the method includes the step of removing urine from the container once said single animal has urinated on the analytical sample collector.

18. A method of collecting an analytical sample of urine from an animal, comprising the step of assembling the kit of claim 13 into the analytical sample collector.

19. The method of claim 18, comprising unsealing the attractant composition.

20. The method of collecting an analytical sample of animal urine of claim 16, wherein the analytical sample collector is clean and/or previously unused.

21. A method of obtaining an analytical sample of animal urine, comprising the step of supplying the analytical sample collector of claim 1 and receiving a sample of urine that has been collected.

22. A method of analysing a sample of urine from an animal, comprising the step of analysing the composition of a urine sample, wherein the urine sample has been collected using the method of claim 16 or wherein the urine sample has been obtained by supplying the analytical sample collector and receiving a sample of urine that has been collected.

23. The method of claim 22, further comprising the step of providing results of the analysis to an interested party.
